(19) United States
(12) Patent Application Publication
(10) Pub. No.: US 2021/0295985 A1
(43) Pub. Date: Sep. 23, 2021

(54) SYSTEM AND METHOD TO PREDICT PATIENTS' DOOR-TO-DOOR HOSPITAL PROCEDURE EVENT TIMELINE

(71) Applicant: KONINKLIJKE PHILIPS N.V., EINDHOVEN (NL)

(72) Inventors: Michael Prokle, Boston, MA (US); Ranjith Naveen Tellis, Tewksbury, MA (US); Sandeep Madhukar Dalal, Winchester, MA (US); Ushanandini Raghavan, Lexington, MA (US); Yuechen Qian, Lexington, MA (US); Douglas B. McKnight, Lexington, MA (US)

(21) Appl. No.: 17/261,720

(22) PCT Filed: Jul. 9, 2019
(86) PCT No.: PCT/EP2019/068326

§ 371 Date: Jan. 20, 2021

Related U.S. Application Data
(60) Provisional application No. 62/700,943, filed on Jul. 20, 2018.

(51) Int. Cl. G16H 40/20; H04W 4/021; G06F 3/0481; G06Q 40/08

(52) U.S. Cl. CPC G16H 40/20; G06Q 40/08; G06F 3/04817; H04W 4/021

(57) ABSTRACT

A non-transitory computer-readable medium stores instructions readable and executable by a mobile device (32) with a display (34) and including at least one electronic processor (35) to perform a patient appointment timeline tracking method (100, 200). The method includes: receiving, via a wireless communication path (36), information at the mobile device about a patient appointment at a medical facility including estimated time information for events in the patient appointment; controlling the display to display a timeline (40) of the events in the patient appointment, the events being displayed as icons (42), the displayed time-line including the time information for the events; detecting, via one or more user inputs, a selection of one of the icons; and in response to detecting the user inputs, controlling the display to present information related to the selected icon.

### FIELD

The following relates generally to the medical arts, patient appointment tracking arts, mobile application arts, and related arts.

### BACKGROUND

Outpatients scheduled for hospital procedures very often are unsure what they can expect for a given hospital visit, and are unaware of the exact scope and work steps of the visit. Many patients go through a certain procedure for the first time (e.g., magnetic resonance imaging (MRI), ultrasound imaging, and so forth) and use the referring physician, personal network or their own research as informational sources ahead of the visit. This information is often only provided at a high level, and may be incomplete or wrong when not coming from a medical professional. Information from such sources may also not be specific to the particular hospital and laboratory with which the appointment is made, and patient check-in, preparation, and examination procedures can vary greatly between medical facilities and even between different laboratories in the same hospital. Information from the referring physician is more likely to be accurate and specific to the patient's appointment, but might be communicated only verbally and is easily forgotten. Hence, on the day of a given hospital visit, a patient does not necessarily have a clear picture of what to expect for a particular hospital visit and the following patient journey through the hospital.

In addition to the little information that the patient has on hand, a patient workflow often has substantial complexity and uncertainty. This makes it even more difficult for the patient to prepare for the visit. For example, a patient is usually given a fixed appointment time a long time in advance, and this appointed time is not adjusted to account for changes in resource availability or other changes and the patient is not given an updated notice.

The status quo of outpatient visits to hospitals is not customer friendly and lacks information, education and predictability. For patients and hospitals, this can be difficult for several reasons. Hospital visits are difficult to plan for outpatients, and the fixed scheduling does not account for patients' time due to the little visibility into the expected duration of the visit, wait times and the expected end of their stay. A lack of visibility in workflow changes affects hospitals and patients. For hospitals, changing workflow conditions are often not well communicated to the patient and schedule adjustments are seldom data-driven. For patients, a delay in patient arrival is not communicated to the hospital and the impact for patient and hospital is unclear. Moreover, limited prior knowledge of the upcoming procedures and/or the equipment used in those procedures tends to create patient anxiety, which can discourage patients from making their appointments (leading to higher instances of no-shows) or unnecessarily extend the length of the procedure due to patient errors (e.g., an MRI scan may have to be repeated due to movement by the patient during the scan). This directly translates into significant hospital department cost. Furthermore, patients may underestimate or fail to understand the importance of a hospital visit or procedure on their health outcome, again tending to lead to increased no-shows. The patient workflow is also typically not efficient. Expensive resources may be blocked or occupied in activities that can be performed in advance of a visit through a web-based application. These include patients' time to fill forms or simple education activities that can be provided to the patient beforehand (e.g., educational videos, questionnaires, FAQs etc.). In addition, patients may fail to comply with pre-procedure instructions (e.g., fasting for a specified time interval prior to a colonoscopy) and hospital staff have only limited ability to remind patients before arriving for the procedure. Patient non-compliance with pre-procedure instructions can lead to an inability to perform the procedure to obtain clinically useful medical data, or to acquisition of medical data of compromised quality and clinical usefulness. In extreme cases, patient non-compliance can lead to a misdiagnosis. For example, if a patient with orders to fast nonetheless consumes a large quantity of sugar prior to drawing blood for a blood sugar test, a misdiagnosis of diabetes could result.

The following discloses new and improved systems and methods to overcome these problems.

### SUMMARY

In one disclosed aspect, a non-transitory computer-readable medium stores instructions readable and executable by a mobile device with a display and including at least one electronic processor to perform a patient appointment timeline tracking method. The method includes: receiving, via a wireless communication path, information at the mobile device about a patient appointment at a medical facility including estimated time information for events in the patient appointment; controlling the display to display a timeline of the events in the patient appointment, the events being displayed as icons, the displayed timeline including the time information for the events; detecting, via one or more user inputs, a selection of one of the icons; and in response to detecting the user inputs, controlling the display to present information related to the selected icon.

In another disclosed aspect, a patient appointment timeline tracking system includes at least one electronic processor; and a non-transitory storage medium storing instructions readable and executable by the at least one electronic processor to perform a patient appointment timeline tracking method. The method includes: determining current time information for events of a patient appointment with events including two or more of the group consisting of: an inbound transport; a check-in; a wait time; a preparation and education; an examination; a recovery; and an outbound transport; generating a timeline of the events of the patient appointment including the current time information; and communicating the timeline including the current time information to a mobile device via a wireless communication path.

In another disclosed aspect, a patient appointment timeline tracking method includes: at a medical server, computing a timeline of events in a patient appointment using current information on at least one of a patient and a medical laboratory retrieved by the medical server; wirelessly transmitting the timeline of events in the patient appointment from the medical server to a mobile device; at the mobile device, displaying the timeline of events in the patient appointment, the events being displayed as icons, the icons include two or more of the group consisting of: an inbound travel icon; a check-in icon; a wait time icon; a preparation and education icon; an examination icon; a recovery icon; and an outbound icon; and keeping the displayed timeline current by repeating the computing of the timeline of events, the wireless transmitting, and the displaying.

One advantage resides in providing a mobile electronic device for enhancing patient understanding of upcoming appointments.

Another advantage providing a system including a mobile electronic device and medical server supplying real-time updates to a patient regarding appointments.

Another advantage resides in providing such a device and system with an intuitive user interface displaying a timeline of the events in the patient appointment with events being displayed as user-selectable icons for accessing further information.

Another advantage resides in expediting patient appointments by reducing delays due to unpreparedness and lack of communication.

Another advantage resides in more efficient patient appointments which can save hospitals time and money.

Another advantage resides in ensuring patients receive pre-appointment information and instructions.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a workflow schedule monitoring system according to one aspect;

FIG. 2 shows an exemplary timeline generated by the system of FIG. 1; and

FIGS. 3-6 show exemplary flow chart operations of the system of FIG. 1.

### DETAILED DESCRIPTION

The following relates to a mobile device designed to enhance the experience of a patient receiving an outpatient procedure, such as an MRI examination. In existing setups, assistance to the patient is typically limited to setting up a scheduled appointment for the procedure, possibly being handed printed material on the procedure, and/or a phone reminder. The disclosed mobile device, which may for example be implemented as a cellphone, tablet computer, or so forth running an application program (“app”) in communication with a backend hospital server provides the patient with current information ahead of the appointment, including more detailed scheduling information that is updated in real-time. The disclosed mobile device employs an intuitive user interface in which the various events or phases of the medical appointment process, such as drive-in, check-in, waiting period, patient preparation/education, the actual medical examination (which may for example be a physical examination, imaging examination, therapeutic session such as a radiation therapy session, or so forth), and recovery period are represented by a timeline including icons for each event or phase. The user (i.e. patient) may select an icon from the timeline to bring up further information about the event. In another example, the user interface can display appointment scheduling options. The appointment scheduling options can include ad-hoc options (such as what imaging center has availability at the moment displaying the real-time wait, or traditional options such as scheduling days in advanced for a reserved spot).

In one illustrative embodiment, the mobile device running the app provides an intuitive user interface which presents a timeline of user-selectable icons. The icons represent time phases of the procedure or appointment, e.g. arrival transport, check-in, waiting, patient prep, examination, recovery, and departure transport phases. The mobile device running the app is in communication with a backend server maintained by the hospital or a service provider that retrieves relevant information from the laboratory scheduling system, patient's EMR, staffing and/or RTLS information, equipment log information, current traffic and weather data proximate to the transport times, or so forth. This information is processed by a simulation & analytics engine to generate up-to-date time projections for the various time phases of the procedure, so as to update these times on the timeline for current information.

The icons of the timeline presented by user interface are, in the illustrative embodiment, designed as user selectable buttons that provide contextual information. For example, if the user selects the “Wait” icon a few days before the scheduled procedure it may provide information such as an image of the waiting room and a currently projected waiting time. The same button selected on the day of the procedure and during the waiting period may return more detailed information such as the number of patients ahead and a current waiting time projection, possibly along with information on the MRI technician who will be performing the procedure (e.g. a portrait photograph of the technician). In another example, entertainment or education options (e.g., on-demand videos) can be provided to the user's mobile device during the waiting period. After the waiting period has passed the Wait icon may be removed, or retained but grayed out, or retained and active to show the actual wait time.

The buttons representing transport to and from the medical facility may provide information such as recommended modes of transportation (e.g. one or more call buttons for taxi service, Uber, Lyft, or another transportation service, links to public transportation maps and schedules, or so forth), currently estimated drive time (which may depend on the mode of transportation) and recommended leave time, and so forth.

The Check-in icon when selected prior to the day of the procedure may provide general information such as an explanation of the check-in procedure and photographs of the check-in desk or other relevant images. On the day of the procedure (or, optionally, even more precisely after the GPS indicates the patient is at the hospital), the check-in button may provide turn-by-turn walking directions to the check-in desk. The examination button is similarly contextual, e.g. providing links to informational videos showing the MRI procedure and safety information from the patient's perspective prior to the day of the examination or more specific information on the particular MRI to perform the procedure on the day of the procedure, a specific reminder to remove all metallic objects, or so forth. The check-in button may also optionally allow for recordation of biometric information such as fingerprints, facial recognition, or so forth which may be used to expedite check-in.

The Recovery icon provides informational text about after-effects, recovery time, or so forth on the days preceding examination day. During the recovery phase this button may bring up relaxing music, and optionally may provide real-time machine-patient dialogs via which the patient can report concerning symptoms and receive assurance that these are normal; or, if the patient-reported symptoms are abnormal they can be reported to laboratory staff via the app. Any available sensors may also be incorporated for assessing patient condition. For example, the patient may be asked to read presented text and the built-in cellphone microphone used to record the patient's voice and perform analyses to detect slurring, response delay and/or incoherency, or other indications of patient distress, and report these to laboratory staff as appropriate. The Recovery button may optionally continue to provide real-time recovery guidance even after the patient has returned home. (The Departure Transport button may optionally be removed or greyed out after GPS indicates the patient is back home so that the Recovery button is thereafter the rightmost active button).

In another example embodiment, the timeline optionally further includes an Exam Results button. Such a button when pressed prior to availability of the results would present generic information about the type of information the examination will provide. After the results become available they may be accessible via the Exam Results button. The advisability of providing this button, and more particularly of providing the actual examination results, depends upon the type of examination and other factors such as the preferences of the patient's physician. For example, the “Exam Results” button may be preferably not included in the case of a timeline for an examination which may present a finding of a terminal condition; whereas, an “Exam Results” button may be usefully provided in the case of a throat culture examination to determine whether a pediatric patient has Streptococcal pharyngitis (strep throat). Similarly, providing raw MRI images may be more confusing than informational for the patient. One contemplated implementation of the optional “Exam Results” button is to make its inclusion a configuration option set by the medical facility, or set for a particular type of examination, and/or configurable by the doctor. Another option would be to present only a written physician's report which such is available, or to contact the patient to setup a follow-up appointment with the physician to discuss the results.

The content brought up by the various icons of the disclosed timeline may change over time as new information becomes available and/or as the workflow progresses. In some embodiments, the icons include badges, i.e. graphical indicators superimposed on the icon such as at an upper right-hand corner of the icon, which appear when an icon contains new information that has not yet been accessed by the patient. In this way, the user (i.e. patient) can see at a glance which icon(s), if any, contain new or updated information. When the patient selects such an icon the new or updated information is displayed, and thereafter the badge is no longer displayed on that icon unless/until further new or updated information comes in.

An appointment checklist may also be provided, which lists tasks the patient should perform prior to the procedure, such as providing appointment confirmation within 48 hours of the appointment, and/or filling out a medical questionnaire, both of which are preferably done via the app. The checklist may also provide information fasting restrictions, informational videos to be viewed prior to the procedure (optionally time-locked to ensure viewing in some specified timeframe, e.g. within three days of the procedure so as to ensure the video is consumed close to the time of the procedure; these may be the same videos accessible via the Exam button), and so forth. Optionally, alarms and/or notifications may be provided, e.g. an alarm and/or notification when the fast period commences.

Any or all of these buttons, checklists, or other UI aspects may optionally be patient-specific. For example, the arrival and departure transport buttons may be specific to the mode of transportation employed by the patient. A patient with cognitive limitations may be provided with additional or different instructional content, may receive additional reminders, or so forth. A pediatric patient may have content and information tailored for a child.

The app may optionally also interact with other applications. In particular, if the patient is to be driven by a family member or friend, the driver may have his/her own app providing current pickup time recommendations and traffic/drive time information. Likewise, if the patient is tracked by GPS this information may be sent by the app to the laboratory so that they can update their schedule based on whether the patient will be arriving on-time.

In some illustrative embodiments, the app runs on a generic mobile device, e.g. a cellphone or tablet computer, with the app configuring the mobile device to connect with the hospital server via a wireless connection (e.g. WiFi or 4G), retrieve up-to-date appointment information, and present the information in accordance with the timeline-based UI. In a contemplated variant embodiment, a dedicated wireless handheld device may be provided to the patient which runs the app. An advantage of this arrangement is that the dedicated device may optionally include dedicated hardware such as vital sign sensors enabling the app to monitor the patient's physiological condition. The system further includes the hospital server executing machine-readable instructions in order to collect and/or generate the up-to-date appointment information. In some embodiments this includes implementing a simulation and analytics engine that generates at least a portion of the up-to-date appointment information by modeling the workflow of the laboratory and/or other aspects such as travel times.

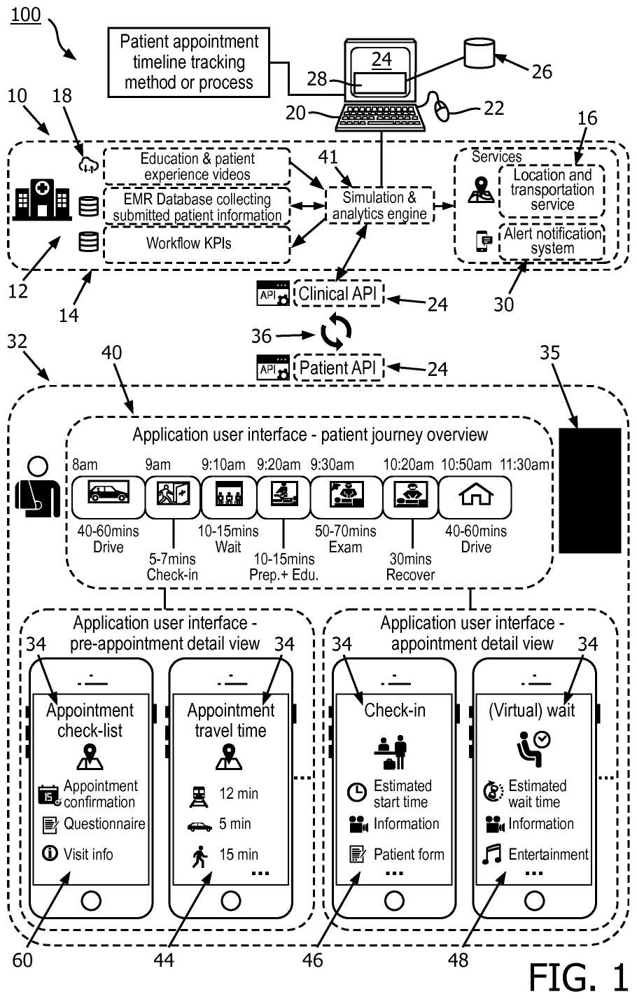

With reference to FIG. 1, an illustrative workflow schedule monitoring system 10 is shown. As shown in FIG. 1, the system 10 includes a first database 12, a second database 14, a real-time location service (RTLS) device 16, a cloud-based server 18, and at least one electronic processor 20. The first database 12 is configured to store patient information such as patient personal information, patient test results, patient vital signs, patient appointment time, and so forth. In some examples, the first database 12 can be an electronic medical record (EMR) database, an electronic health record (EHR) database, a cardiovascular information system (CVIS), a combination of two or more of these, or so forth. The second database 14 is configured to store workflow information for the hospital, the specific laboratory to which the outpatient appointment is directed, or a combination thereof, the workflow information including, by way of non-limiting illustrative example, workflow schedule process time stamps, staffing schedules clinical resource availability, real-time patient and staff locations (e.g., via GPS data); and one or more key performance indicators (KPIs) as metrics of the quality of the appointment schedule. By way of non-limiting illustrative example, the KPIs may, for example, include one or more of: total predicted patient waiting time for all patients scheduled for procedures; maximum waiting time predicted for any single patient scheduled for a procedure (e.g., if patients A, B, C, D, and E have respective predicted waiting times of 2 min, 5 min, 25 min, 7 min, and 4 min, then the maximum waiting time KPI value would be 25 min); total operating costs; staff costs; total staff overtime; and/or so forth. These illustrative KPIs are each preferably minimized, but the optimization can alternatively be formulated as a maximization problem. The optimization figure of merit (i.e. objective function) can include a weighted combination of several KPIs, with weighting values chosen to scale the values to comparable units (e.g., time-based KPIs and cost-based KPIs are made comparable by suitable scaling) and to weight the relative importance of the various KPIs.

The RTLS device 16, if provided or accessed, generates position data of the patient (and optionally also the staff and mobile medical equipment that may be assigned to the laboratory on an occasional basis), and stores this data in the first database 12 (or, alternatively, the RTLS may be accessed on an as-needed basis to identify the location of a patient or staff person). In some examples, the RTLS device 16 includes a GPS device configured to obtain GPS location data of the patient and store this data in the first database 12. By way of non-limiting illustration, one example of a suitable RTLS is an RFID-based RTLS employing radio frequency identification (RFID) tags worn by staff, on a patient bracelet, disposed on or in tracked equipment, or so forth and tracked by RFID tag readers placed at strategic locations around the hospital or other medical facility. An RTLS tags database stores tag-subject assignments enabling association of RFID tags with the tagged individuals or equipment, and an electronic map of the hospital or other medical facility identifies the location based on which RFID tag reader picks up the RFID tag (or, in a more advanced embodiment, detection of the RFID tag by two or three RFID tag readers enables more precise location by way of triangulation). For the purposes of the workflow scheduling, it may be sufficient for the RTLS 16 to be used to classify each patient or staff member as one of (1) not in the hospital; (2) in the hospital but not at the laboratory to which the appointment is directed; or (3) at the laboratory. In the case of mobile medical equipment, typically only categories (2) or (3) will apply. In some embodiments, the RTLS 16 can be used to determine if a staff member is available. For example, if the location of each staff member is known, then the locations can be compared to the planned schedule to infer staff utilization (e.g., staff member A is scheduled for a procedure on patient B with staff member C). In another example, the location information can be used for historical timestamps (e.g., nurse A is utilized for X minute for procedure Y), which can be stored in the first database 12. It will be further appreciated that a combination of GPS and RTLS can be useful in maximizing locational information, as the GPS accurately identifies location on the road (e.g. when driving to or from the hospital) but is less accurate or inoperative in the confines of the hospital building; whereas the RTLS provides locational information inside the hospital.

The cloud-based server 18 may include or access further database(s) (not shown) configured to store information such as education videos and patient experience videos. These videos include information that the patient needs to know before an appointment, including a description of the procedure, any pre-appointment requirements the patient must perform (e.g., fasting before a procedure), recovery information after the procedure, and so forth. These videos are transferred from the cloud-based server 18 to the patient.

The at least one electronic processor 20 comprises a computer, a workstation, a cloud-based server computer, or other electronic data processing device. In some examples, the workstation 20 can include typical components, such as at least one user input device (e.g., a mouse, a keyboard, a trackball, and/or the like) 22, and a display device or application program interface (API) 24. It should be noted that these components can be variously distributed. For example, the electronic processor 20 may include a local processor of a workstation terminal and the processor of a server computer that is accessed by a workstation terminal. In some embodiments, the display device 24 can be a separate component from the computer 18. The workstation 18 can also include one or more databases or non-transitory storage media 26. The various non-transitory storage media 12, 14, 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth. They may also be variously combined, e.g. a single server RAID storage may store both databases 12, 14. The display device 24 is configured to display a graphical user interface (GUI) 28 including one or more fields to receive a user input from the user input device 22.

In some embodiments, the system 10 also includes an alert generation device 30 configured to generate an alert based on an adjustment of a proposed workflow schedule. For example, the alert generation device 30 can include a device to generate a Messaging Service (MS) text message, a Short Messaging Service (SMS), an alert in a web-based program such as Microsoft Outlook, and so forth in order to inform a patient of, for example rescheduling of the patient's appointment time, information for the patient's review before the appointment, and so forth. In some embodiments the patient may be given the option to accept or reject the rescheduling, in which case the system will not update the schedule to reflect the rescheduling unless and until the patient accepts by way of a return text message. Additionally or alternatively, badges may be superimposed on icons of the displayed timeline user interface to indicate icons with updated content.

The system 10 is in wireless communication with a mobile device 32 that is in the possession of the patient. The mobile device 32 can be, for example, a smart cellphone or tablet computer running a general-purpose mobile operating system such as iOS or Android and operating a mobile application program (“app”) such as an iOS app or Android app, respectively, by which the patient receives information from the system 10 and, in some embodiments, communicates information back to the system 10. In some embodiments, the mobile device 32 may be a dedicated wireless handheld device that is provided to the patient by the hospital and which runs the app. The mobile device 32 includes a display screen 34 and at least one electronic processor 35. The mobile device 32 and the system 10 are in wireless communication with each other via a wireless communication path 36 (illustratively shown as arrows), e.g. in accord with an Application Programming Interface (API) 24 that defines the methods of communication between the app running on mobile device 32 and the system 10.

The system 10 and/or the mobile device 32 is configured to perform a patient appointment timeline tracking method or process 100. A non-transitory storage medium stores instructions which are readable and executable by the at least one electronic processor 20, 35 and to perform disclosed operations including performing the patient appointment timeline tracking method or process 100. The operations of the method or process 100 may be variously divided between the mobile device 32 and the backend system 10. As the latter has substantially greater processing power, it is typically advantageous to perform computationally intensive operations such as modeling at the system 10, while the mobile device 32 performs less computationally intensive tasks, and/or tasks that benefit from local processing, such as presenting the timeline-based UI and receiving and processing user inputs. In some examples, the method 100 may be performed at least in part by cloud processing (e.g., the server 18 may be a cloud-based server or cloud computing resource). The instructions which are executed to perform the workflow schedule monitoring method or process 100 may be viewed as implementing (i) a simulation and analytics engine 38 and (ii) controlling the display 34 of the mobile device 32 to display a current timeline 40 of events of a patient appointment that is transmitted from the simulation and analytics engine to the mobile device.

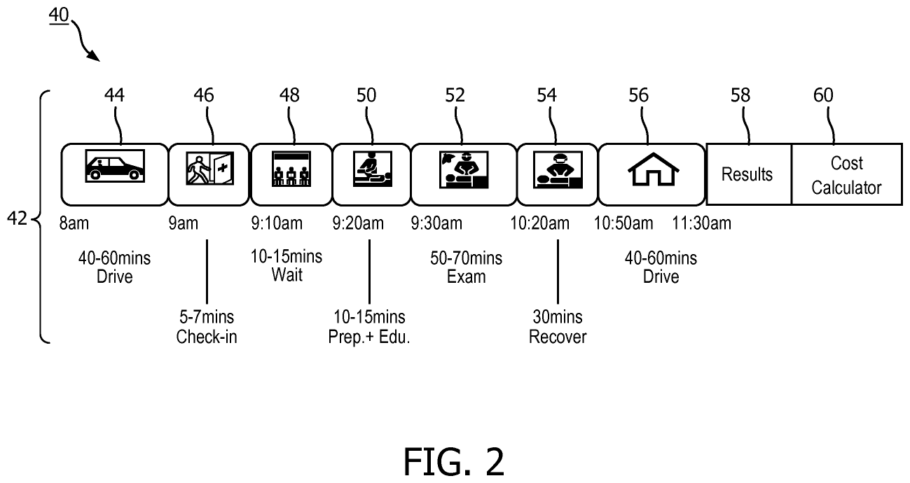

FIG. 2 shows an example of the timeline 40 of events (i.e. phases) of a patient appointment in which the events include at least estimated time information for the events in the appointment. The events are shown as user-selectable icons 42 that can be selected, for example, by a user tapping one of the icons with a finger. The illustrative icons 42 include: an inbound transport icon 44; a check-in icon 46; a wait time icon 48; a preparation and education icon 50; an examination icon 52; a recovery icon 54; and an outbound transport icon 56. A given implementation may include a sub-set of these (e.g., two or more icons 42), and/or may include further icon(s). For example, in some embodiments, the icons 42 further include a results icon 58, in which the user can view the results of their procedure. As further illustrative variants, the recovery icon 54 may be omitted in the case of patient appointments in which there is no required recovery time, such as a procedure for acquiring a throat culture, or a blood draw procedure. Alternatively, the recovery icon 54 may be retained but its selection will merely provide an informational message such as “This procedure requires no recovery time or follow-up actions. You will be able to leave immediately after completion of the procedure.” In some embodiments, the timeline is personalized for the particular outpatient and the particular appointment, or the workflow steps of the timeline can be customized in a particular department or hospital. Thus, for example, the recovery icon 54 may be omitted in timelines of appointments for procedures that have no recovery time or follow-up actions, but included in timelines of appointments for procedures that have a recovery time and/or follow-up actions. The illustrative timeline 40 is oriented horizontally with events from left-to-right corresponding to events generally in a time sequence (although time ordering is not necessarily strict, for example, the patient may elect to review the preparatory information via selection of the icon 50 prior to check-in). In other embodiments the timeline may have a different orientation, e.g. vertical with events running from top-to-bottom corresponding to events generally in a time sequence.

In another embodiment, the icons 42 can further include a cost calculator icon 60. For example, the cost calculator icon 60 can provide information to a user related to an expected range of cost and reimbursement based on patients undergoing similar procedures having similar benefit (i.e., insurance) carriers. Thus, the user can determine an amount of out-of-pocket expenses for the procedure. In another example, the cost calculator icon 60 can provide information to a user related to ways or tips on how to maximize benefits under the patient's insurance plan (i.e., to reduce the out-of-pocket expenses for the user) by providing insights on how to best comply with the procedure according to the benefit's available under the user's plan.

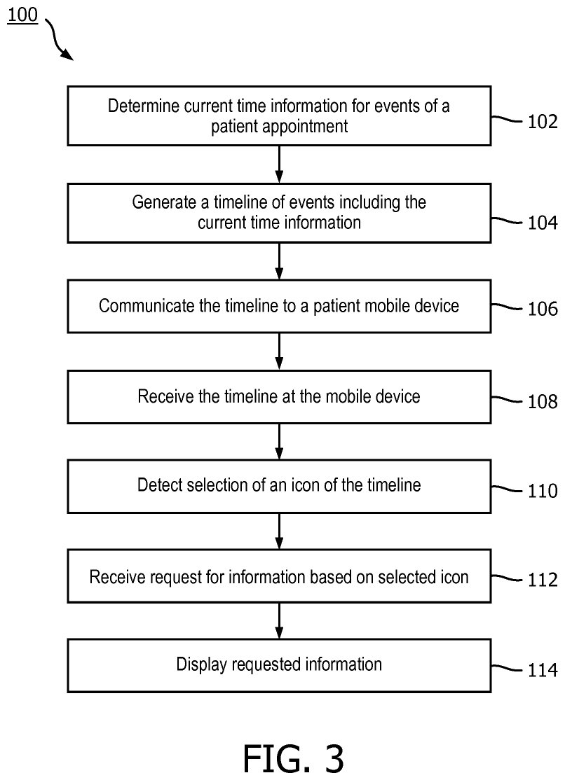

The system 10 and the mobile device 32 operate in tandem to perform the patient appointment timeline tracking method 100. With reference to FIG. 3, an illustrative embodiment of the patient appointment timeline tracking method 100 is diagrammatically shown as a flowchart. At 102 (e.g. performed by a simulation and analytics engine 41 in the illustrative logical module architecture of FIG. 1), the at least one electronic processor 20 is programmed to determine current time information for events of a patient appointment. In one embodiment, the simulation and analytics engine 41 is programmed to receive a current patient location from the RTLS 16. For example, the RTLS 16 is programmed to track a location of the mobile device 32 via a GPS module of the mobile device, and thus determine a position of the patient. The RTLS 16 then transmits this location to the simulation and analytics engine 41. Based on the transmitted location, the simulation and analytics engine 41 is also programmed to determine travel-related current information based on a computed distance between the transmitted location of the mobile device 32 and the location of the medical facility. From the current positon of the patient and the travel-related information, the simulation and analytics engine 41 is programmed to determine at least one current time information.

In some embodiments, the simulation and analytics engine 41 is programmed to predict a future patient workflow using statistical process distributions derived from historic patient flow time stamp information. The location data collected by the real-time location service 16 is used by the simulation and analytics engine 41 to further refine the predicted workflow based on actual observed patient arrival, procedure time lengths and resource availability (e.g., nurse). This allows the simulation and analytics engine 41 to constantly update and improve process time forecasts (e.g., patient procedure end) based on the real-time information. In one example, the timeline 40 can be updated in real-time for additional procedures ordered in an initial examination (e.g., extra laboratory procedures are requested upon an initial examination).

In another embodiment, the simulation and analytics engine 41 is programmed to receive current information on a medical laboratory of the patient appointment (e.g., availability, wait time, and so forth). From this, the simulation and analytics engine 41 is programmed to simulate workflow of the medical laboratory using the current information to determine the current time information including at least a wait time at the medical laboratory.

At 104, the at least one electronic processor 20 is programmed to generate the timeline 40 of the events of the patient appointment including the current time information. For example, the simulation and analytics engine 41 is programmed to generate the icons 42 to generate the timeline 40. At 106, the at least one electronic processor 20 is programmed to communicate the timeline 40 including the current time information to the mobile device 32 via a wireless communication path 36, where it is displayed on the display screen 34 of the mobile device.

At 108, the mobile device 32 is configured (e.g., via API 24) to receive, via the wireless communication path 36, information about the patient appointment at a medical facility including estimated time information for events in the patient appointment. The information can be the generated timeline 40 of icons 42. The processor 35 of the mobile device 32 then controls the display screen to display the timeline 40. In some examples, the receiving of information at the mobile device 32 about the patient appointment includes receiving updates for the time information for events, and the display of the timeline 40 includes updating the displayed timeline to include the updated time information for the events.

At 110, the mobile device 32 is configured to detect, via one or more user inputs (e.g., finger taps on a touch-sensitive display 34 of the mobile device 32), a selection of one of the icons 42. At 112, the simulation and analytics engine 41 is programmed to receive a request for information based on the selected icon 42. For example, the mobile device 32 is configured to transmit an indication of detection of a user input via the wireless communication path 36 to the system 10 as a request for information regarding the selected icon 42. In response to receiving the request, the simulation and analytics engine 41 is programmed to generate the requested information, and transmit this information to the mobile device 32. At 114, in response to detecting the user inputs, the processor 35 is programmed to control the display 34 to present information received from the system 10 related to the selected icon 42. In some instances, the mobile device may handle a user input without invoking the system 10—for example, user selection of the recovery icon 54 in cases in which the procedure requires no recovery or follow-up may bring up a default text, stored at the mobile device 32, indicating that the patient will be able to go home immediately and that no follow-up steps are required.

In one example, the received information includes information on available modes of transportation to the medical facility including transport time estimates for each mode. Upon detection of selection of the inbound transport icon 44, the processor 35 transmits this detection as a request for transport time estimate information to the system 10. In response to receiving the request, the simulation and analytics engine 41 is programmed to retrieve information including location of the patient, traffic information on routes between the patient and a medical facility, and weather information from the second database 14. An estimated time for arrival for the patient is calculated by the simulation and analytics engine 41 based on the retrieved information. Alternatively, the simulation and analytics engine 41 may receive the estimated time for arrival based on a travel time estimate received from a third party GPS-based traffic navigation service. The estimated time for arrival is communicated via the wireless communication path 36 to the mobile device 32. The processor 35 is the programmed to control the display 34 to present selectable options representing different modes of transportation for the patient to arrive by the estimated time for arrival.

In another example, the received information includes information on a wait time based on a number of patient in a waiting room, a check-in procedure and directions information. Upon detection of selection of the wait time icon 48, the processor 35 transmits this detection as a request for wait time information to the system 10. In response to receiving the request, the simulation and analytics engine 41 is programmed to retrieve information including a number of patients waiting in a wait room of a medical facility, and an average time of wait from the second database 14. An estimated wait time for the patient is calculated by the simulation and analytics engine 41 based on the retrieved information. This may employ any suitable approach, e.g. simulation of the workflow or an empirical approach such as referencing a look-up table storing (x,y) data pairs where “x” denotes the number of persons in the waiting room and “y” denotes the typical wait time for that number “x” of waiting persons. The estimated wait time is communicated via the wireless communication path 36 to the mobile device 32. The processor 35 is the programmed to control the display 34 to present the estimated wait time. In another example, the wait time can be estimated by the simulation and analytics engine 41 using other additional information, such as a nature of a procedure to be performed on individual patients in a workflow (e.g., a check-up procedure, an imaging procedure, and so forth), medical personnel availability, medical equipment availability, real-time schedule updates, and so forth.

In some embodiments, upon selection of the wait time icon 48, information related to a delay cost can be shown on the display 34. For example, with the wait times being estimated, there will be a likelihood that a number of patients would wait to the last possible moment to arrive. This can create a significant problem as one patient delay actually causes delay to all patients thereafter and the resources of the hospital are so expensive to waste. As such, a forecast to the patient of the cost (e.g., in the form of additional wait time) for late arrival to the required check-in time. In some examples, different degrees of costs can be applied to a profile of the user. For example, if the patient is tracked by the RTLS 16 and is forecasted to be on time, but then the traffic patterns change, (e.g. an accident, change in weather, etc.) the patient can be penalized to a lesser extent than a patient who merely shows up late or not at all.

In a further example, the received information includes information on results from a procedure performed on the patient. Upon detection of selection of the results icon 58, the processor 35 transmits this detection as a request for results information to the system 10. In response to receiving the request, the simulation and analytics engine 41 is programmed to retrieve information including the results on the procedure performed on the patient from the first database 12. The patient's results are communicated via the wireless communication path 36 to the mobile device 32. The processor 35 is the programmed to control the display 34 to present the patient's results.

It will be appreciated by one of skill in the art that the above-described operations can be similarly performed based on a selection of the other icons 42 in the timeline 40 (e.g., the check-in icon 46; the preparation and education icon 50; the examination icon 52; the recovery icon 54; and the outbound transport icon 56). In another example, the received information includes information a check-in procedure and directions information. Upon detection of selection of the check-in icon 46, the display 34 of the mobile device 32 is configured to display or present an explanation of the check-in procedure and directions from an entrance of the medical facility to a check-in desk. In yet another example, the received information includes information on an explanation of preparation procedures and education materials related to a procedure to be performed on the patient. Upon detection of selection of the preparation and education icon 50, the display 34 of the mobile device 32 is configured to display or present the explanation of preparation procedures and the education materials related to the procedure to be performed on the patient. In another example, the received information includes information including an explanation of a procedure to be performed on the patient and an estimated time of completion of the procedure. Upon detection of selection of the examination icon 52, the display 34 of the mobile device 32 is configured to display or present the explanation of the procedure to be performed on the patient and the estimated time of completion of the procedure. In a further example, the received information includes after effects of a procedure to be performed on the patient, recovery time, and entertainment information including music and reading material. Selection of the preparation and education icon 50 may also solicit information from the patient, for example by presenting an electronic questionnaire form for the patient to fill out electronically (e.g. by selecting options from drop-down lists, radial selection buttons, typing in text via a soft keyboard, and/or so forth) and then sending the completed questionnaire information back to the server 10 which forwards it to the doctor, laboratory, or other appropriate recipient. Upon detection of selection of the recovery icon 54, the display 34 of the mobile device 32 is configured to display or present the aftereffects of a procedure to be performed on the patient, and the recovery time and output the reading material and the music (e.g., via a speaker (not shown) of the mobile device). In yet another example, the received information includes information including location of the patient, traffic information on routes between the medical facility and a home of a patient; and weather information. Similar to the operations performed upon detection of selection of the inbound transport icon 44, upon detection of the outbound transport icon 56, the display 34 of the mobile device 32 is configured to display or present an estimated time transit to the home the patient based on the location of the patient, the traffic information on routes between the a medical facility and the home of a patient; and the weather information, and also to provide selectable options for transport for the patient. In some examples, a survey request following the outpatient visit can be sent to the mobile device 32 to be filled out by the patient.

In some embodiments, the system 10 is configured to send materials to the mobile device 32 for the patient to review before the medical procedure. The system 10 is configured to transmit, via the wireless communication path 36, information for review by a patient before a procedure to be performed on the patient and a checklist 60 listing the materials for the patient to review before the procedure from the cloud-based server 18 to the mobile device 32. This information and the checklist 60 is displayed on the display screen 34. The user can “tap” selections on the checklist 60 to indicate that the patient has reviewed a particular selection. Upon selecting one of the selections in the checklist 60, the system 10 receives an indication that a portion of the transmitted information has been viewed. The simulation and analytics engine 41 is programmed to update the checklist upon receiving the indication and transmit the updated checklist to the mobile device 32, where it is displayed on the display screen 34.

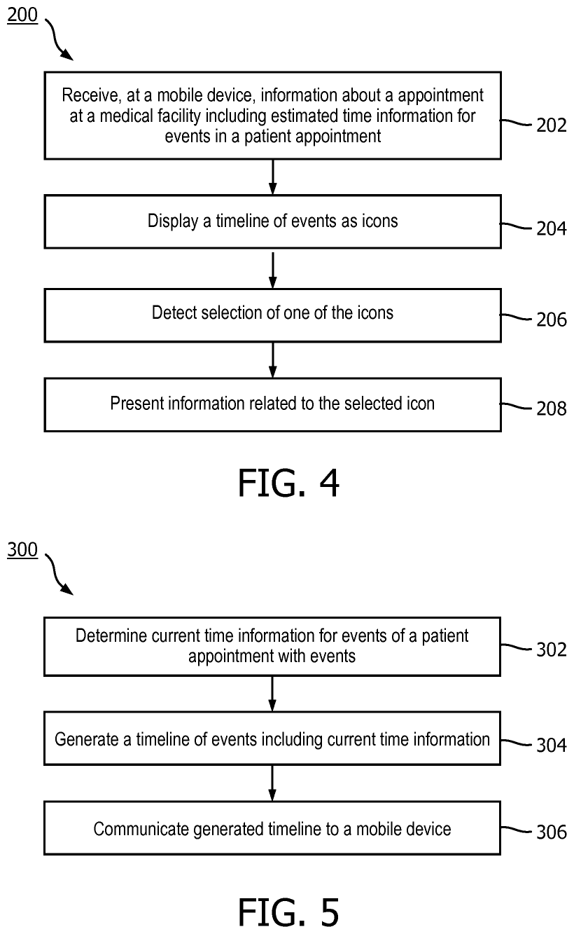

With reference to FIG. 4, another illustrative embodiment of the patient appointment timeline tracking method 200 is diagrammatically shown as a flowchart. At 202, information about a medical appointment at a medical facility including estimated time information for events in the patient appointment is received at the mobile device 32 via the wireless communication path 36. At 204, the display 34 is configured to display the timeline 40 of the events in the patient appointment in which the events are displayed as the icons 42 and the displayed timeline includes the time information for the events. At 206, the mobile device 32 is configured to detect a selection of one of the icons 42 via one or more user inputs. At 208, in response to detecting the user inputs, the display 34 is configured to present information related to the selected icon 42.

With reference to FIG. 5, another illustrative embodiment of the patient appointment timeline tracking method 300 is diagrammatically shown as a flowchart. At 302, the at least one electronic processor 20 is programmed to determine current time information for events of a patient appointment with events including at least an inbound transport 44, a check-in 46, a wait time 48, a preparation and education 50, an examination 52, a recovery 54, and an outbound transport 56. At 304, the at least one electronic processor 20 is programmed to generate a timeline 40 of the events of the patient appointment including the current time information. At 306, the at least one electronic processor 20 is programmed to communicate the timeline 40 including the current time information to the mobile device 32 via the wireless communication path 36.

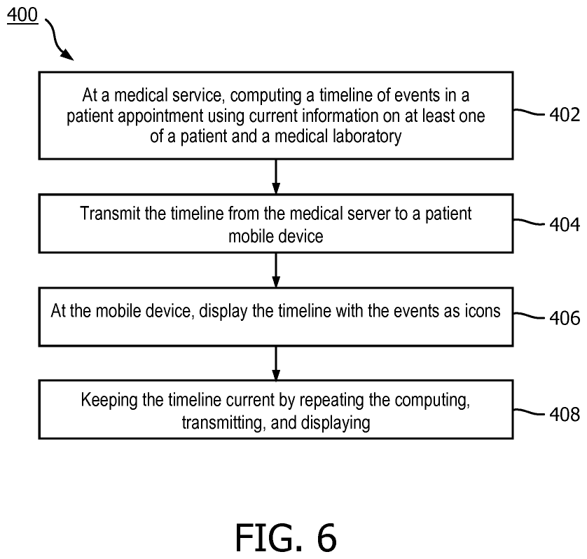

With reference to FIG. 6, another illustrative embodiment of the patient appointment timeline tracking method 400 is diagrammatically shown as a flowchart. At 402, at a medical server or system 10, a timeline 40 of events in a patient appointment is computed using current information on at least one of a patient and a medical laboratory retrieved by the medical server. At 404, the timeline of events in the patient appointment is transmitted from the medical server 10 to a mobile device 32. At 406, at the mobile device 32, the timeline 40 is displayed on a display screen 34 of the mobile device in which the events are as icons 42 including at least an inbound travel icon 44; a check-in icon 46; a wait time icon 48; a preparation and education icon 50; an examination icon 52; a recovery icon 54; and an outbound icon 56. At 408, the displayed timeline is kept current by repeating the computing of the timeline of events, the wireless transmitting, and the displaying.

As another contemplated capability, the app running on the mobile device may be capable of pushing notifications to the user (i.e. patient). This can be implemented using the “notifications” capability of the iOS or Android operating system, and can be usefully leveraged for example to notify the patient of the start of a preparatory fasting period, to remind the patient that he or she has not yet reviewed preparatory materials, or so forth.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A non-transitory computer-readable medium storing instructions readable and executable by a mobile device with a display and including at least one electronic processor to perform a patient appointment timeline tracking method, the method comprising:
receiving, via a wireless communication path, information at the mobile device about a patient appointment at a medical facility including estimated time information for events in the patient appointment;
controlling the display to display a timeline of the events in the patient appointment and an estimated wait time, the events being displayed as icons, the displayed timeline including the time information for the events, and the estimated wait time based on one or more of a count of patients waiting in a wait room of the medical facility, an average time of wait, procedures to be performed on individual patients in a workflow, availability of medical personal, availability of medical equipment, or real-time schedule updates;
detecting, via one or more user inputs, a selection of one of the icons; and
in response to detecting the user inputs, controlling the display to present information related to the selected icon.

2. The non-transitory computer-readable medium of claim 1, wherein the icons include at least two or more of the group consisting of: an inbound transport icon; a checkin icon; a wait time icon; a preparation and education icon; an examination icon; a recovery icon; and an outbound transport icon, wherein the examination icon provides links to informational videos showing an MRI procedure and safety information from a patient's perspective.

3. The non-transitory computer-readable medium of claim 1, wherein the receiving of information at the mobile device about the patient appointment includes:
receiving updates for the time information for events, and the display of the timeline includes updating the displayed timeline to include the updated time information for the events.

4. The non-transitory computer-readable medium of claim 3, wherein the received information includes information on available modes of transportation to the medical facility including transport time estimates for each mode, and upon detection of selection of the inbound transport icon, the method further includes:
controlling the display to present selectable options representing different modes of transportation for the patient to arrive by the estimated time for arrival.

5. The non-transitory computer-readable medium of claim 1, wherein the received information includes information a check-in procedure and directions information and, upon detection of selection of the check-in icon, the method further includes:
controlling the display to present an explanation of the check-in procedure and directions from an entrance of the medical facility to a check-in desk.

6. (canceled)

7. (canceled)

8. The non-transitory computer-readable medium of claim 1, wherein the received information includes information on an explanation of preparation procedures and education materials related to a procedure to be performed on the patient and, upon detection of selection of the preparation and education icon, the method further includes:
controlling the display to present the explanation of preparation procedures and the education materials related to the procedure to be performed on the patient.

9. The non-transitory computer-readable medium of claim 1, wherein the received information includes information including an explanation of a procedure to be performed on the patient and an estimated time of completion of the procedure and, upon detection of selection of the examination icon, the method further includes:
controlling the display to present the explanation of the procedure to be performed on the patient and the estimated time of completion of the procedure.

10. The non-transitory computer-readable medium of claim 1, wherein the received information includes aftereffects of a procedure to be performed on the patient, recovery time, and entertainment information including music and reading material and, upon detection of selection of the recovery icon, the method further includes:
controlling the display to present the aftereffects of a procedure to be performed on the patient, and the recovery time; and
outputting the music and reading material.

11. The non-transitory computer-readable medium of claim 1, wherein the received information includes information including location of the patient, traffic information on routes between a medical facility and a home of a patient; and
weather information and, upon detection of selection of the outbound transport icon, the method further includes:
calculating an estimated time transit to the home of the patient based on the location of the patient, the traffic information on routes between the a medical facility and the home of a patient; and the weather information; and
controlling the display to present the estimated time for transit.

12. The non-transitory computer-readable medium of claim 11, wherein the method further includes:
determining a delay cost for the patient when the patient is determined to be late for a scheduled appointment time; and
controlling the display to present the generated delay cost.

13. The non-transitory computer-readable medium of claim 12, wherein the delay cost is determined based on the location of the patient, the traffic information, and the weather information.

14. The non-transitory computer-readable medium of claim 1, wherein the icons further include a results icon, and upon detection of selection of the results icon, the method further includes:
retrieving information including results from a procedure performed on the patient;
controlling the display to display the retrieved information.

15. The non-transitory computer-readable medium of claim 1, wherein the icons further include a cost calculator icon, and upon detection of selection of the cost calculator icon, the method further includes:
controlling the display to present information related to an expected range of cost and reimbursement based on patients undergoing similar procedures having similar benefit carriers.

16. The non-transitory computer-readable medium of claim 1, wherein the icons further include a cost calculator icon, and upon detection of selection of the cost calculator icon, the method further includes:
controlling the display to present information related to ways on how to maximize benefits under the patient's insurance plan to reduce the out-of-pocket expenses for the user by providing insights on how to best comply with the procedure according to the benefit's available under the user's plan.

17. The non-transitory computer-readable medium of claim 1, wherein the method further includes:
receiving, from a medical professional, information for review by a patient before a procedure to be performed on the patient and a checklist listing the materials for the patient to review before the procedure;
controlling the display to display the received information; and
updating the checklist upon receiving a user input that the patient reviews the received information.

18. A patient appointment timeline tracking system, comprising:
at least one electronic processor; and
a non-transitory storage medium storing instructions readable and executable by the at least one electronic processor to perform a patient appointment timeline tracking method including:
determining current time information for events of a patient appointment with events including at least two or more of the group consisting of: an inbound transport; a check-in; a wait time; a preparation and education; an examination; a recovery; and an outbound transport;
generating a timeline of the events of the patient appointment including the current time information;
communicating the timeline including the current time information to a mobile device via a wireless communication path;
receiving a current patient location from a real time location system;
determining at least some current time information based on the current patient location and travel-related current information; and
determining a future patient workflow based on the determined current time information and an actual patient arrival time, procedure times, and resource availability.

19. (canceled)

20. The system of claim 18, wherein the determining of current time information includes:
receiving current information on a medical laboratory of the patient appointment; and
simulating workflow of the medical laboratory using the current information to determine the current time information including at least a wait time at the medical laboratory.

21. The system of claim 18, wherein the patient appointment timeline tracking method further includes:
receiving, from the mobile device via the wireless communication path, a request for travel information; and
in response to receiving said request:
retrieving information including location of the patient, traffic information on routes between the patient and a medical facility, and weather information;
calculating an estimated time for arrival for the patient based on the retrieved information; and
communicating the estimated time for arrival to the mobile device via the wireless communication path.

22. The system of claim 18, wherein the patient appointment timeline tracking method further includes:
receiving, from the mobile device via the wireless communication path, a request for a wait time; and
in response to receiving said request:
retrieving information including a number of patients waiting in a wait room of a medical facility, and an average time of wait;
calculating an estimated wait time for the patient based on the retrieved information; and
communicating the estimated wait time to the mobile device via the wireless communication path.

23. (canceled)

24. (canceled)

25. A patient appointment timeline tracking method, comprising:
at a medical server, computing a timeline of events in a patient appointment using current information on at least one of a patient and a medical laboratory retrieved by the medical server;
wirelessly transmitting the timeline of events in the patient appointment from the medical server to a mobile device;
at the mobile device, displaying the timeline of events in the patient appointment, the events being displayed as icons, the icons including two or more of the group consisting of: an inbound travel icon; a check-in icon; a wait time icon; a preparation and education icon; an examination icon; a recovery icon; and an outbound icon; and
keeping the displayed timeline current by repeating the computing of the timeline of events, the wireless transmitting, and the displaying.
